SEQUENCE LISTING (4 sequences)

SEQ ID NO: 1 - DNA, 20 bp, Artificial Sequence
  Synthetic oligonucleotide to act as a PCR forward primer to amplify RNF213 gene fragment.
ttccagaacgtccagcaagt

SEQ ID NO: 2 - DNA, 20 bp, Artificial Sequence
  Synthetic oligonucleotide to act as a PCR reverse primer to amplify RNF213 gene fragment.
acagtcctggtcctgtcaga

SEQ ID NO: 3 - DNA, 18 bp, Artificial Sequence
  Synthetic oligonucleotide to act as a probe to detect amplified RNF213 gene fragment.
ctccatcaaaggcttcct

SEQ ID NO: 4 - DNA, 18 bp, Artificial Sequence
  Synthetic oligonucleotide to act as a probe to detect amplified RNF213 gene fragment.
ctccatcagaggcttcct

(19) United States
(12) Patent Application Publication
(10) Pub. No.: US 2025/0059597 A1
(43) Pub. Date: Feb. 20, 2025

(54) PRESERVATION METHOD FOR PCR REAGENT, AND PCR TEST METHOD

(71) Applicant: SHIMADZU CORPORATION, Kyoto (JP)

(72) Inventor: Daisuke KAWAKAMI, Kyoto (JP)

(73) Assignee: SHIMADZU CORPORATION, Kyoto (JP)

(21) Appl. No.: 18/699,147

(22) PCT Filed: Jun. 29, 2022
(86) PCT No.: PCT/JP2022/025912

§ 371 Date: Jun. 24, 2024

(30) Foreign Application Priority Data
Nov. 11, 2021 (JP) 2021-184214

(51) Int. Cl. C12Q 1/686; C12Q 1/6806; C12Q 1/6827; C12Q 1/6848

(52) U.S. Cl. CPC C12Q 1/686; C12Q 1/6806; C12Q 1/6827; C12Q 1/6848; C12Q 2600/156

(57) ABSTRACT

The present preservation method is a preservation method for a PCR reagent, the method includes the steps of (1) mixing all of a first composition containing DNA polymerase, a second composition containing a primer, and a third composition containing a buffer liquid to prepare a reaction liquid; and (2) refrigerating the reaction liquid, wherein the first composition, the second composition, and the third composition are accommodated in different containers.

### CROSS REFERENCE TO PRIOR APPLICATION

This application is a National Stage Patent Application of PCT International Patent Application No. PCT/JP2022/025912 (filed on Jun. 29, 2022) under 35 U.S.C. § 371, which claims priority to Japanese Patent Application No. 2021-184214 (filed on Nov. 11, 2021), which are all hereby incorporated by reference in their entirety.

### REFERENCE TO A SEQUENCE LISTING SUBMITTED VIA PATENT CENTER

The content of the ASCII text file of the sequence listing named “2024-10-29_1875-0033_SequenceListing_rev.txt”, which is 1,329 bytes in size, was created on Oct. 28, 2024 and electronically submitted via Patent Center on Oct. 29, 2024, is incorporated herein by reference in its entirety.

### TECHNICAL FIELD

The present invention relates to a preservation method for a PCR reagent, and a PCR test method.

### BACKGROUND ART

In recent years, the number of strokes has been increasing more and more, and cerebral infarction accounts for about 70% to 80% of the causes thereof. The cerebral infarction is a disease in which a part of a blood vessel in a brain is clogged, a sufficient blood flow does not reach a brain cell ahead of the clogged blood vessel, and the brain cell dies. The cerebral infarction is mainly classified into three types: atherothrombotic cerebral infarction (a symptom in which a large blood vessel in the brain is clogged by arteriosclerosis), lacunar infarction (a symptom in which a small blood vessel deep in the brain is clogged), and cardiogenic cerebral embolism (a symptom in which blood clots formed in other parts, such as the heart, flow through the bloodstream and become clogged). For a patient who has developed cerebral infarction, the type of cerebral infarction is diagnosed together with the location of the onset of the cerebral infarction, and treatment is performed according to the type of cerebral infarction diagnosed. Usually, the type of cerebral infarction is diagnosed by confirming a CT/MRI image or an image in which a road map of DRR images (digitally reconstructed radiographs) is displayed on a fluoroscopic image at the time of treatment.

In addition, since the onset frequency of the cerebral infarction is particularly high in Japan as compared to Western countries, the presence of genetic factors different from Western countries has been pointed out, and it has been reported that RNF213 gene p. R4810K polymorphism (a genetic polymorphism in which arginine at position 4810 of a protein encoded by a gene called RNF213 gene is changed to lysine) is a sensitive gene for atherothrombotic cerebral infarction. Therefore, recently, attempts have been made to use not only image information in the related art but also information of RNF213 gene p. R4810K polymorphism in order to diagnose the type of cerebral infarction for a patient who has developed cerebral infarction, and it is expected to lead to improvement in diagnosis accuracy. In order to determine whether or not a patient who has developed cerebral infarction has an RNF213 gene p. R4810K polymorphism, it is common to collect blood of the patient and detect a variant of the RNF213 gene p. R4810K polymorphism by a real-time PCR method (refer to Patent Document 1).

### PRIOR ART DOCUMENT

### Patent Document

Patent Document 1: Japanese Patent Application Laid-Open No. 2020-92660

### SUMMARY OF THE INVENTION

### Problems to be Solved by the Invention

Meanwhile, in an acute phase of cerebral infarction, when a patient is transported to a hospital, blood sampling, CT/MRI, and image interpretation are performed, and treatment is immediately started as soon as the onset position, type, and the like of cerebral infarction are confirmed. In particular, in the cerebral infarction, as the start of initial treatment is delayed, the degree of damage to the brain becomes more serious, and thus treatment as soon as possible is required. Therefore, in order to use information on the RNF213 gene p. R4810K polymorphism for determination of the cerebral infarction type in the acute phase, it is required to complete the test by the PCR method in a short time (within about 1 hour).

However, in the test by the conventional PCR method, the time from specimen collection to completion of PCR exceeds one hour. One of the causes for this time is complicated work in the preparation stage until the test by the PCR method is performed. Specifically, a reagent kit includes, for example, three or more kinds of reagents such as an enzyme-containing reagent, a primer/probe-containing reagent, and a buffer liquid-containing reagent. These are dispensed in a desired amount immediately before PCR to prepare a PCR reaction liquid, the PCR reaction liquid is mixed with a specimen such as blood, and the mixed solution is set in a PCR apparatus. In addition, in order to extract a nucleic acid from a sample (for example, blood) collected from a patient, a pretreatment step of adding a dedicated treatment liquid to denature a protein and liberate the nucleic acid is also required. Furthermore, from the viewpoint of preventing false negative or false positive diagnosis, positive control or negative control may be required, and in this case, work of mixing a PCR reaction liquid with the control is also added. As described above, in the conventional method, since the dispensing and mixing operations of the reagents are complicated, much time is required in the preparation stage before the PCR apparatus is set, and as a result, the time of the entire PCR test from the specimen collection to the completion of PCR exceeds one hour. Therefore, a reduction in the time for PCR test is required.

An object of the present invention is to provide a method capable of completing a PCR test in a short time.

### Means for Solving the Problems

A preservation method for a PCR reagent according to a first embodiment of the present invention includes the steps of: (1) mixing all of a first composition containing DNA polymerase, a second composition containing a primer, and a third composition containing a buffer liquid to prepare a reaction liquid; and (2) refrigerating the reaction liquid, in which the first composition, the second composition, and the third composition are accommodated in different containers.

A PCR test method according to the first embodiment of the present invention includes the steps of performing the preservation method according to the first embodiment; preparing a liquid from which a nucleic acid is extracted by adding a pretreatment liquid to a specimen; mixing the liquid from which the nucleic acid is extracted with the reaction liquid; and performing PCR on the mixed reaction liquid.

### Effects of the Invention

According to the preservation method for a PCR reagent and the PCR test method of the first embodiment, the PCR test can be completed in a short time.

### BRIEF DESCRIPTION OF THE DRAWINGS

FIG. 1 illustrates a schematic diagram of a preservation method (preparing step, mixing step, and refrigeration step) according to a first embodiment.

FIG. 2 illustrates a schematic diagram of a PCR step according to the first embodiment.

FIG. 3 illustrates PCR detection results of Example 1, in which FIG. 3A illustrates the PCR result for a first positive control in a first PCR tube, FIG. 3B illustrates the PCR result for a second positive control in a second PCR tube, FIG. 3C illustrates the PCR result for a third positive control in a third PCR tube, and FIG. 3D illustrates the PCR result for a specimen-containing reaction liquid in a fifth PCR tube. In each PCR result, a horizontal axis represents the number of PCR cycles, a vertical axis represents fluorescence intensity, a solid line in a graph represents fluorescence intensity by a fluorescently labeled probe (FAM) that binds to a wild-type base sequence, and the broken line in the graph represents luminescence intensity by a fluorescently labeled probe (ROX) that binds to a mutant-type base sequence.

FIG. 4 illustrates PCR results of Example 2, FIG. 4A illustrates the result of the first PCR tube, FIG. 4B illustrates the result of the second PCR tube, FIG. 4C illustrates the result of the third PCR tube, and FIG. 4D illustrates the result of the fifth PCR tube.

FIG. 5 illustrates PCR results of Example 3, FIG. 5A illustrates the result of the first PCR tube, FIG. 5B illustrates the result of the second PCR tube, FIG. 5C illustrates the result of the third PCR tube, and FIG. 5D illustrates the result of the fifth PCR tube.

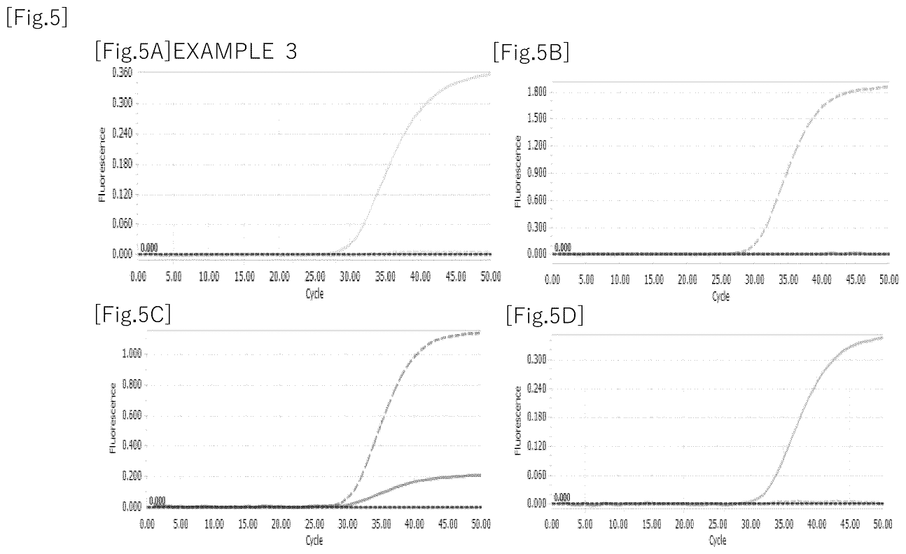

FIG. 6 illustrates PCR results of Example 4, FIG. 6A illustrates the result of the first PCR tube, FIG. 6B illustrates the result of the second PCR tube, FIG. 6C illustrates the result of the third PCR tube, and FIG. 6D illustrates the result of the sixth PCR tube.

FIG. 7 illustrates PCR results of Reference Example 1, FIG. 7A illustrates the result of the first PCR tube, FIG. 7B illustrates the result of the second PCR tube, FIG. 7C illustrates the result of the third PCR tube, and FIG. 7D illustrates the result of the sixth PCR tube.

FIG. 8 illustrates a schematic diagram of a preservation method (preparing step, mixing step, and refrigeration step) performed in each Example.

FIG. 9 illustrates a schematic diagram of the PCR step performed in each Example.

FIG. 10 illustrates PCR results of PCR results of Example 5, FIG. 10A illustrates the result of the first PCR tube, FIG. 10B illustrates the result of the second PCR tube, FIG. 10C illustrates the result of the third PCR tube, and FIG. 10D illustrates the result of the sixth PCR tube.

### MODE FOR CARRYING OUT THE INVENTION

### 1. First Embodiment

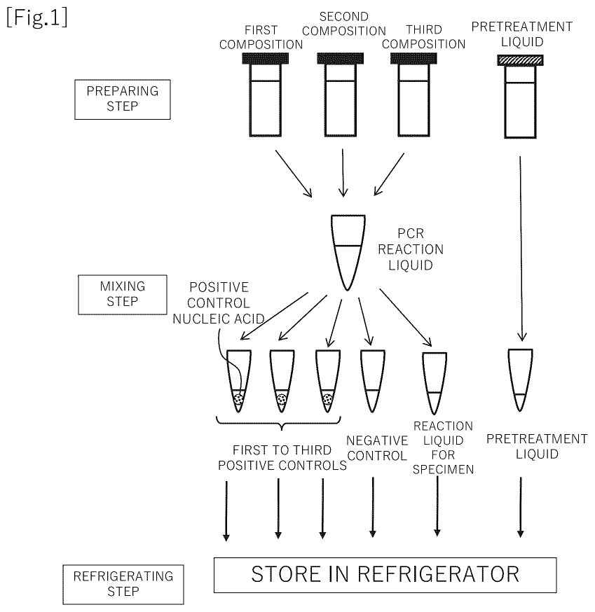

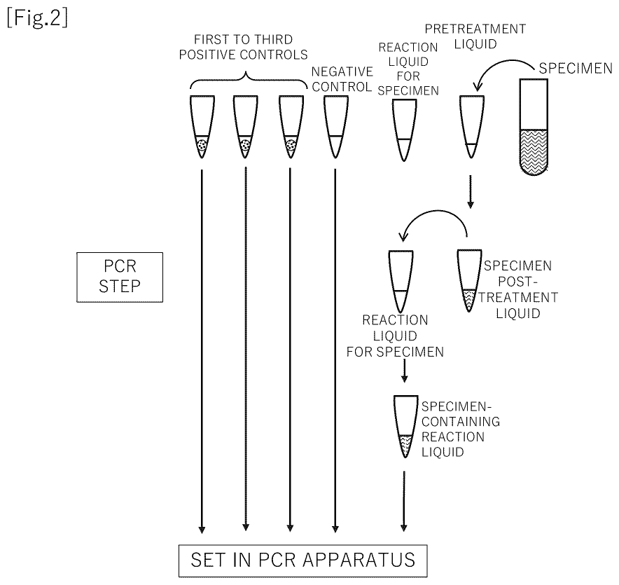

A first embodiment of the present invention is a test method by polymerase chain reaction (PCR), which includes a preservation method and a PCR step in order as shown in FIGS. 1 and 2. In the preservation method according to the first embodiment, (1) a preparation step and (2) a refrigeration step are sequentially performed. In the PCR test method of the first embodiment, a case where a test target of the PCR test, that is, the nucleic acid to be amplified is the RNF213 gene p. R4810K polymorphism will be described as a representative, but the test target is not limited to this polymorphism.

The RNF213 gene p. R4810K polymorphism (hereinafter, it may be abbreviated as “the present polymorphism”) is a polymorphism in which the arginine at position 4810 of the protein encoded by the RNF213 gene is changed to lysine. The present polymorphism is a single nucleotide polymorphism in which the 14576 base of the RNF213 gene is guanine (G) in the wild type, whereas it is adenine (A) in the mutant type. In the present polymorphism, there are three types of “mutant-type/mutant-type” homozygote, “wild-type/wild-type” homozygote, and “mutant-type/wild-type” heterozygote.

### (Preparation Step)

In this step, the first composition containing DNA polymerase, the second composition containing a primer, and the third composition containing a buffer liquid are all mixed to prepare a reaction liquid. This step includes, for example, (i) a preparing step of preparing a first composition, a second composition, and a third composition accommodated in different containers, and (ii) a mixing step of mixing all of these first to third compositions in order.

(i) In the preparing step, a reagent set for a reaction liquid is prepared. Preferably, a pretreatment liquid is further prepared in addition to a reagent set for a reaction liquid.

A reaction liquid reagent set essentially includes a DNA polymerase, a primer, and a buffer liquid, and preferably further includes a probe and a dNTP mix. Specifically, the reagent kit includes at least a first composition containing DNA polymerase, a second composition containing a primer, and a third composition containing a buffer liquid, and these compositions are separately accommodated in three containers (for example, a vial or the like).

The first composition accommodated in the first container contains a DNA polymerase, and is preferably a first liquid composition containing a DNA polymerase and a solvent.

The DNA polymerase is, for example, a thermostable DNA polymerase derived from thermophilic bacteria, and specific examples thereof include a Taq DNA polymerase, a Tth DNA polymerase, a KOD DNA polymerase, a Pfu DNA polymerase, and variants thereof. In addition, from the viewpoint of reducing non-specific amplification by the DNA polymerase, a hot start DNA polymerase such as BIOTAQ (registered) DNA polymerase may be used.

The solvent of the first composition or the second composition is not limited, and examples thereof include aqueous solvents such as purified water, physiological saline, and a buffer liquid, organic solvents such as glycerol, and mixed solvents thereof.

The second composition accommodated in the second container contains a primer, and is preferably a second liquid composition containing a primer, a probe, and a solvent.

The primer is an oligonucleotide that hybridizes (binds) to the present polymorphism to be amplified and amplifies the present polymorphism, and is preferably an oligonucleotide that amplifies a base sequence containing a genetic mutation corresponding to the present polymorphism. The base sequence containing the genetic mutation corresponding to the present polymorphism is a base sequence containing the 14576 base (guanine or adenine) of the RNF213 gene. By selecting a primer to be hybridized into the present polymorphism, the PCR reaction liquid mixed with the first to third compositions is hardly affected by the reaction with the coexisting DNA polymerase in the refrigerated preservation state, and PCR can be performed even after preservation.

Primers usually consist of a primer pair of a forward primer and a reverse primer. The base length of each primer pair is preferably, for example, 10 bases or more and 30 bases or less. Specific examples of the primer pair include oligonucleotides represented by SEQ ID NO: 1 and SEQ ID NO: 2.

(SEQ ID NO: 1)
(Forward) 5′-TTCCAGAACGTCCAGCAAGT-3′
(SEQ ID NO: 2)
(Reverse) 5′-ACAGTCCTGGTCCTGTCAGA-3′

The probe is an oligonucleotide probe that can be hybridized in the present polymorphism, and is preferably an oligonucleotide fluorescently labeled probe. As the oligonucleotide fluorescently labeled probe, one kind alone or a combination of two or more kinds can be adopted, and preferably, a combination of an oligonucleotide fluorescently labeled probe that hybridizes to a mutant-type base sequence containing a genetic mutation corresponding to the present polymorphism and an oligonucleotide fluorescently labeled probe that hybridizes to a wild-type base sequence corresponding to the mutant-type base sequence is mentioned. The mutant-type base sequence containing the genetic mutation corresponding to the present polymorphism is a base sequence containing the 14576 base when the 14576 base of the RNF213 gene is a mutant-type (adenine) base. The wild-type base sequence corresponding to the mutant-type base sequence is a base sequence containing the 14576 base when the 14576 base of the RNF213 gene is a wild-type (guanine) base. As a result, two types of the mutant type and the wild type can be distinguished from each other and simultaneously detected. In addition, the probe that binds to the present polymorphism is less likely to be affected by a reaction with a coexisting DNA polymerase in a refrigerated preservation state of the PCR reaction liquid mixed with the first to third compositions, and PCR can be reliably performed even after preservation.

The base length of such a probe is preferably, for example, 10 bases or more and 30 bases or less. Specific examples of the combination of probes include oligonucleotide fluorescently labeled probes having the base sequences set forth in SEQ ID NO: 3 and SEQ ID NO: 4.

Probe that binds to a mutant type base sequence:
(SEQ ID NO: 3)
5′-CTCCATCAAAGGCTTCCT-3′
Probe that binds to a wild-type base sequence:
(SEQ ID NO: 4)
5′-CTCCATCAGAGGCTTCCT-3′

In the oligonucleotide fluorescently labeled probe, a reporter (fluorescent dye) and a quencher are modified at the end of the base sequence. Examples of the type of oligonucleotide fluorescently labeled probe include a hydrolysis probe (for example, TaqMan probe), a molecular beacon probe, and a cycling probe, and a hydrolysis probe is preferable. The hydrolysis probe is a probe in which the 5′ end and the 3′ end are modified with a reporter or a quencher. Specifically, a reporter is bound to the 5′ end of the probe represented by SEQ ID NO: 3 or 4, and a quencher is bound to the 3′ end.

Examples of the modified reporter include FAM (6-carboxyfluorescein), ROX (6-carboxy-X-rhodamine), TET (6-carboxy-4,7,2′,7-Tetrachloro fluorescein), HEX (4,7,2,4,5,7-hexachloro-6-carboxyfluorescein), Cy3 (Cyanine dye), and Cy5 (Cyanine dye). When the two types of oligonucleotide labeled probes are used, a combination of reporters whose fluorescent wavelength ranges do not overlap may be selected.

The quencher to be modified is appropriately determined depending on the reporter, and examples thereof include TAMRA (6-carboxytetramethylrhodamine), BHQ-1 ([(4-(2-Nitro-4 methyl-phenyl)-azo)-yl-((2-methoxy-5 methyl-phenyl)-azo)]-anilline), BHQ-2([(4-(1-Nitro-phenyl)-azo)-yl-((2,5-dimethoxy-phenyl)-azo)]-anilline), Dabcyl(4-[[4-Dimethylaminol-phenyl]-azo]-benzoic acid), and Eclipse (4-[[2-Chloro-4-nitro-phenyl]-azo]-anilline).

The third composition accommodated in the third container contains a buffer liquid, and is preferably a third liquid composition containing a buffer liquid and a dNTP mix.

Examples of the buffer liquid include a phosphate buffer liquid, a Tris buffer liquid, a borate buffer liquid, and a Good's buffer liquid (such as HEPES).

The dNTP mix serves as a substrate for the PCR reaction and is a mixture consisting of dATP (deoxyadenosine triphosphate), dGTP (deoxyguanosine triphosphate), dCTP (deoxycytidine triphosphate), and dTTP (thymidine triphosphate).

The third composition may further contain a substance that suppresses the action of the PCR inhibitor. Specifically, a substance that binds to a biologically negative charge substance (for example, certain sugars, dyes) adsorbed to DNA polymerase or a biologically positive charge substance (for example, certain proteins) adsorbed to DNA and neutralizes the PCR inhibitory action of the negative charge substance or the positive charge substance may be contained. Examples of the buffer liquid containing such a substance include Ampdirect (registered trademark, manufactured by Shimadzu Corporation), which is a reagent for gene amplification, and the like. As a result, the nucleic acid contained in a specimen post-treatment liquid together with the contaminants can be subjected to PCR without purification, so that PCR test can be performed in a short time.

The third composition may further contain additives such as magnesium chloride (MgCl₂) and potassium chloride (KCl) from the viewpoint of further activating the enzyme of DNA polymerase.

The first to third compositions may contain various additives in addition to the above components for a desired purpose. Suitable blending proportions of the components of the first to third compositions can be appropriately allocated by those skilled in the art.

The reagent set for a reaction liquid preferably further includes a positive control. The positive control prevents false negative diagnosis where PCR does not occur properly and is erroneously determined as negative. A positive control contains a positive control nucleic acid.

The positive control nucleic acid is a present polymorphism, and may be any of (a) a wild-type/wild-type homozygote, (b) a mutant-type/mutant-type homozygote, and (c) a mutant-type/wild-type heterozygote. Preferably, these three kinds are separately prepared, that is, three kinds of positive control nucleic acids are prepared. As a result, it is possible to easily confirm which zygote the test target corresponds to. The positive control nucleic acid is accommodated in a container different from the containers for the pretreatment liquid and the first to third compositions. If there are two or three positive control nucleic acids, they are each accommodated in separate containers. The control nucleic acid may be artificially synthesized, or may be previously extracted and amplified from another specimen. The positive control is accommodated in a container different from the containers for the first to third compositions.

The pretreatment liquid prepared together with the reagent set for a reaction liquid is a proteolytic liquid for extracting (eluting) a nucleic acid contained in a membrane such as an envelope in a biological specimen collected from a subject. For example, when blood is used as the specimen, the pretreatment liquid is a blood lysate, and specifically contains a blood lysing component such as a surfactant. Surfactants include anionic surfactants, cationic surfactants, amphoteric surfactants, and nonionic surfactants. From the viewpoint of solubility and reaction inactivity with other components, the anionic surfactants and the nonionic surfactants are preferable, and the anionic surfactants are more preferable. Examples of the anionic surfactant include sodium dodecyl sulfate, ammonium dodecyl sulfate, sodium cholate, alkyl benzene sulfonate, and alkyl carboxylate. Examples of the nonionic surfactant include polyoxyethylene sorbitan monolaurate (Tween (registered trademark) 20), polyoxyethylene sorbitan monooleate (Tween (registered trademark) 80), and polyoxyethylene p-t-octylphenol (Triton (registered trademark) X-100).

When the pretreatment liquid is used as a blood lysate, an anticoagulant such as ethylenediaminetetraacetic acid or citric acid may be further preferably contained. In addition, in the pretreatment, a proteolytic enzyme such as proteinase may be contained as necessary instead of the surfactant, but it is preferable not to contain the protease.

The solvent of the pretreatment liquid is not limited, and examples thereof include aqueous solvents such as purified water, physiological saline, and a buffer liquid, organic solvents such as glycerol, and mixed solvents thereof. The pretreatment liquid is accommodated in a container different from the containers for the first to third compositions.

(ii) In the mixing step, the first composition, the second composition, and the third composition are mixed. That is, these three compositions are accommodated in one container. Thus, the PCR reaction liquid (hereinafter, may be abbreviated as “reaction liquid”) is prepared.

After mixing, the reaction liquid is dispensed into a test container. A plurality of test containers are preferably prepared. Specifically, in addition to a specimen test container (an example of the second test container), a positive control test container (an example of the first test container) and a negative control test container are prepared in order to confirm false negative and false positive. Regarding the positive control test container, preferably, three positive control test containers are prepared in order to be able to measure the types of three zygotes.

Apart of the reaction liquid is dispensed into the specimen test container and the negative control test container. A part of the reaction liquid is dispensed into the positive control test container together with the positive control nucleic acid. That is, (a) the wild-type/wild-type homozygote, (b) the mutant-type/mutant-type homozygote, or (c) the mutant-type/wild-type heterozygote is dispensed into the first, second, or third positive control test container together with the reaction liquid. The amount of each reaction liquid to be dispensed is appropriately determined according to the type of PCR apparatus or nucleic acid, and may be, for example, 5 to 50 μL. The amount of the positive control nucleic acid may be, for example, 0.1 to 10 μL. In addition to these test containers, from the viewpoint of starting the PCR test in a shorter preparation time, a pretreatment test container (an example of a third test container) is also preferably prepared, and the pretreatment liquid is dispensed into the container.

As a result, a specimen test container accommodating only a reaction liquid, a negative control test container accommodating only a reaction liquid, (a) a first positive control test container accommodating a wild-type/wild-type homozygote and a reaction liquid, (b) a second positive control test container accommodating a mutant-type/mutant-type homozygote and a reaction liquid, and (c) a third positive control test container accommodating a mutant-type/wild-type heterozygote and a reaction liquid are prepared.

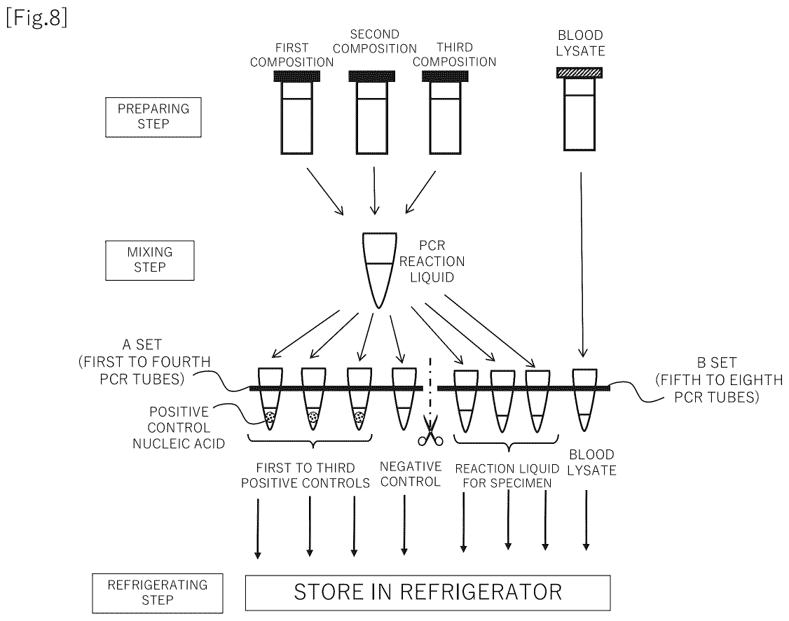

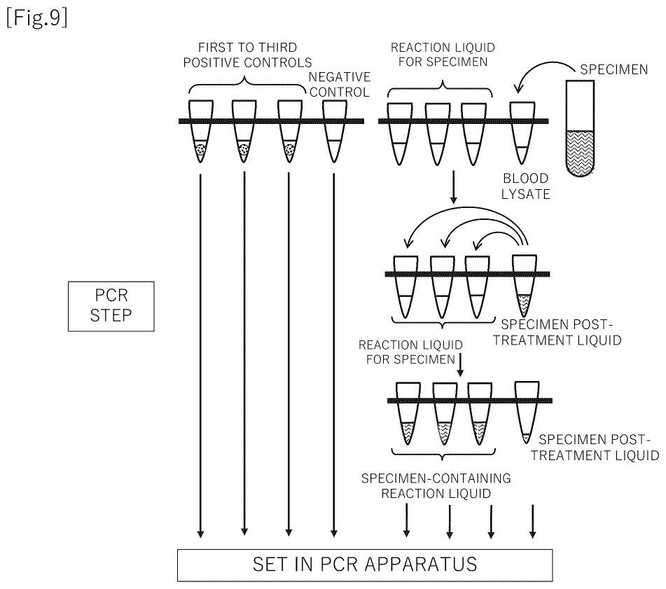

The test container is preferably a container that can be directly set in a PCR apparatus and tested, and examples thereof include a PCR tube. The PCR tube can be procured as a strip tube in which a plurality of PCR tubes are connected. The strip tube is used by individually cutting each tube when PCR is used. By using one strip tube and making the tubes connected to each other into a specimen test container, a positive control test container, and a negative control test container, respectively, the PCR reagent set can be collectively accommodated in one strip tube, so that it is easy to perform management and refrigeration, and the time from the start of operation to the PCR apparatus set can be further shortened. At this time, in terms of management, preferably, the pretreatment liquid is also accommodated in another PCR tube in the same strip tube, and a pretreatment test container in which only the pretreatment liquid is accommodated is also prepared. Furthermore, one strip tube (for example, eight consecutive strip tubes) may be prepared and divided into a plurality of sets (A set, B set), and a positive control test container and a negative control test container may be assigned to one set (for example, A set including four consecutive tubes), and a specimen test container and a pretreatment test container may be assigned to the other set (for example, a B set of four consecutive tubes) (refer to FIGS. 8 and 9). As a result, it is also possible to reduce the risk of contamination of the positive control and the negative control when the specimen is added to the pretreatment test container and stirred or the specimen is dispensed into the specimen test container. The strip tube may be divided from the above viewpoint, but may not be divided.

In mixing the positive control nucleic acid and the reaction liquid, the reaction liquid may be temporarily stored in a refrigerator before mixing. As a result, deterioration due to contact of the positive control nucleic acid with the reaction liquid for a long time can be suppressed, and a decrease in accuracy of PCR can be suppressed. The temporary storage time in this case may be, for example, 60 days or less, and preferably 30 days or less.

### (Refrigeration Step)

In this step, the reaction liquid is refrigerated. Specifically, a specimen test container accommodating only a reaction liquid, a negative control test container accommodating only a reaction liquid, and positive control test containers (a) to (c) accommodating a positive control nucleic acid and a reaction liquid are stored in a refrigerator. At this time, together with these containers, a pretreatment test container storing only the pretreatment liquid may also be stored in the refrigerator.

A refrigerating temperature is, for example, 10° C. or lower, preferably 5° C. or lower, and is, for example, 0° C. or higher. When the temperature exceeds 10° C., the DNA polymerase in the reaction liquid is activated during refrigeration, and the accuracy of PCR after preservation may be reduced. On the other hand, when the temperature is lower than 0° C., the reaction liquid is frozen, so that it may take time to thaw, or each main component such as the positive control nucleic acid may be damaged.

The refrigerating time is, for example, 1 hour or more, preferably 24 hours or more, and is, for example, 60 days or less. If the refrigerating time is too short, for example, the preparation step is performed during treatment for emergency patients such as cerebral infarction, and a simple PCR test method cannot be provided in some cases. On the other hand, when the refrigerating time is too long, components in the reaction liquid may be deteriorated.

The PCR reagent preserved in this step includes at least a reaction liquid (for specimen test) containing a DNA polymerase, a probe, and a buffer liquid in one container, and is preferably a reagent kit including the reaction liquid (for specimen test), a reaction liquid that is the same component as the reaction liquid (for negative control), a reaction liquid containing a positive control nucleic acid (for positive control), and a pretreatment liquid in separate containers.

### (PCR Step)

In this step, PCR is performed using the PCR reagent stored in the refrigeration step. That is, PCR is performed using the PCR reagent set, and a PCR product (amplified nucleic acid) is detected.

First, a specimen containing a nucleic acid (the present polymorphism) which is a test target is collected. The specimen is a biological sample from which nucleic acid can be collected, and is, for example, a body fluid such as blood, cerebrospinal fluid, lymph fluid, saliva, nasal discharge, urine, or feces. Examples thereof include cells or tissues such as hair, nails, skin, and mucous membranes. From the viewpoint of low invasiveness, handleability, and the like, blood is preferable. The blood includes whole blood, plasma, serum, and the like.

Next, as pretreatment, a nucleic acid is extracted from the specimen. Specifically, the specimen is added into the pretreatment test container, and the specimen is mixed with the pretreatment liquid. As a result, a specimen post-treatment liquid in which the nucleic acid is extracted from the specimen is obtained by the dissolving action of the pretreatment liquid. At this time, the specimen may be diluted 2 to 10 times in advance as necessary and then mixed with the pretreatment liquid. In addition, a heat treatment may be performed as necessary. In a case of heating, the heating temperature is, for example, 50° C. or higher and 95° C. or lower, and the heating time is, for example, 3 minutes or more and 10 minutes or less.

Next, the specimen post-treatment liquid is added into the specimen test container. That is, the extracted nucleic acid is mixed with the reaction liquid. As a result, the specimen-containing reaction liquid is prepared in the specimen test container.

Next, the specimen test container is set in a PCR apparatus together with a positive control test container (first to third positive control test containers) and a negative control test container, and PCR is performed.

PCR is performed in several tens of cycles including, as one cycle, (i) a thermal denaturation step of converting double-stranded DNA into single-stranded DNA, (ii) an annealing step of hybridizing a PCR primer and the probe of the present invention to single-stranded DNA, and (iii) an extension step of extending DNA from the PCR primer by a DNA polymerase to form double-stranded DNA. Pre-incubation (for example, 300 to 600seconds at 95° C.) is appropriately performed before the several tens of cycles.

As a specific method of PCR, a real-time PCR method is preferably employed. In the real-time PCR method, the amplification amount of PCR is monitored and analyzed in real time. Examples of the PCR method include an intercalator method and a hydrolysis probe method, and in the first embodiment, a hydrolysis probe method is preferable from the viewpoint that the above-described fluorescently labeled probe can be used to detect a plurality of zygotes.

The number of PCR cycles is, for example, 30 times or more, and is, for example, 50 times or less, preferably 35 times or less. When the lower limit of the number of cycles is 30, the nucleic acid is sufficiently amplified, and the rising of the amplification curve can be confirmed. On the other hand, when the upper limit of the number of cycles is 50, saturation of amplification can be prevented to shorten the measurement time. In particular, by setting the upper limit to 35 times, the rising of the amplification curve can be confirmed, and at the same time, it is possible to perform from specimen collection to completion of PCR in a short time.

The heating temperature and time per PCR cycle are appropriately determined, and it is preferable to set at least the temperature and time at the time of denaturation and the temperature and time at the time of annealing (setting of upper limit temperature and lower limit temperature) to about 5±3 seconds at 95±3° C. and about 15±3 seconds at 60 ±3° C., respectively. As a result, the rising of the amplification curve of the PCR can be confirmed even with a small number of PCR cycles of about 35 times, and the PCR in a short time (for example, about 45 minutes) can also be achieved.

Thereafter, the test result of PCR is confirmed. That is, the presence or absence of the rising of the amplification curve in the specimen-containing reaction liquid is confirmed by monitoring the PCR apparatus, and positive or negative is diagnosed. Specifically, it is determined whether the specimen has a mutant-type base sequence and/or a wild-type base sequence by checking that the amplification curve by the specimen-containing reaction liquid is (a) fluorescence emitted by a fluorescently labeled probe that binds to the mutant-type base sequence or (b) fluorescence emitted by a fluorescently labeled probe that binds to the wild-type base sequence. Then, when the specimen has a mutant-type base sequence, it can be diagnosed as positive, that is, suspected of atherothrombotic cerebral infarction.

At the same time, for each of the positive control (first to third positive controls) and the negative control, the presence or absence of the rising of the amplification curve is confirmed. Specifically, it is confirmed that the rising of the desired amplification curve is observed in each positive control and the rising of the amplification curve is not observed in the negative control, and the success or failure of the false negative and the false positive with respect to the specimen-containing reaction liquid is diagnosed.

A first embodiment is a preservation method for a PCR reagent, the method including the steps of: (1) mixing all first to third compositions to prepare a reaction liquid; and (2) refrigerating the reaction liquid, in which the first to third compositions are accommodated in different containers, respectively. That is, in the preservation method of this step, a PCR reagent composed of a DNA polymerase, a primer, and a buffer liquid accommodated in separate containers is prepared, and these are all mixed and refrigerated. This makes it possible to reduce the time from specimen collection to completion of PCR, particularly the time required for PCR preparation work. Specifically, the refrigerated reaction liquid may be added to the specimen (for example, a specimen to which a pretreatment liquid has been added) from which the nucleic acid is extracted, and set in the PCR apparatus, so that the number of steps of immediately preceding preparation is reduced, and the time for immediately preceding preparation of PCR can be significantly shortened. In addition, since the preserved reaction liquid is refrigerated, the deterioration of the reaction liquid is suppressed, and the rising of the amplification curve of the PCR can be reliably detected even after the refrigeration, and the PCR measurement can be performed. In particular, the PCR reaction liquid contains, as a primer pair and a probe, a primer pair and a probe that bind to the present polymorphism that is difficult to react in a refrigerated state, but does not contain an enzyme having a strong activity such as proteinase, and thus, a reaction between components in the reaction liquid is less likely to occur during refrigerated storage, and the PCR reaction liquid can be preserved for a long period of time in a liquid state in which the components are mixed. Therefore, in the PCR test method including the preservation method according to the first embodiment, it is possible to shorten the time from specimen collection to completion of PCR, for example, within 60 minutes.

In the first embodiment, it is preferable to predict the use of the PCR reagent and perform this preservation method at least by the day before the day when the use is predicted. As an example, a plurality of PCR reagents are stored by the present preservation method, and as soon as the PCR reagents are used (consumed), the PCR reagents are newly added by the number used, and the PCR reagents are stored by the present preservation method. As a result, since a certain number of PCR reagents can be regularly provided, PCR test can be always performed in a short time for an urgent patient.

### 2. Second Embodiment

In the first embodiment, the reaction liquid reagent set preferably includes a positive control, and the positive control is mixed with the reaction liquid. However, for example, the reaction liquid reagent set may not include the positive control. In this embodiment, a preferred example of the PCR reagent to be preserved is, for example, a reagent kit including a reaction liquid (for specimen test), a reaction liquid (for negative control) that is the same component as the reaction liquid, and a pretreatment liquid in separate containers. In the present invention, the first embodiment is preferable from the viewpoint that a false negative can be confirmed and test accuracy is improved.

### 3. Aspects

It is understood by those skilled in the art that the exemplary embodiments described above are specific examples of the following aspects.

(Aspect 1) A method according to one aspect is a preservation method for a PCR reagent, the method including the steps of: (1) mixing all of a first composition containing DNA polymerase, a second composition containing a primer, and a third composition containing a buffer liquid to prepare a reaction liquid; and (2) refrigerating the reaction liquid, in which the first composition, the second composition, and the third composition may be accommodated in different containers. As a result, the PCR test can be performed by a simple operation, so that the PCR test can be completed in a short time. That is, it is not necessary to mix the first to third compositions immediately before PCR, and it is only necessary to mix a refrigerated reaction liquid with a specimen and set the mixture in a PCR apparatus, so that PCR preparation work becomes very simple and processing can be performed in a short time.

(Aspect 2) In the method according to Aspect 1, the PCR reagent may further include a positive control, and in the step (2), a part of the reaction liquid may be accommodated in a first test container together with the positive control, and only a part of the reaction liquid may be accommodated in a second test container, and then the first and second test containers may be refrigerated. As a result, if the first test container including the positive control is set in the PCR apparatus as it is after refrigeration, the PCR test of the positive control can be completed, so that the PCR test that can simultaneously confirm false negatives can be completed in a short time.

(Aspect 3) In the method according to Aspect 1 or 2, the second composition may further contain an oligonucleotide probe. With this, it is possible to reliably perform PCR.

(Aspect 4) In the method according to any one of Aspects 1 to 3, the refrigerating temperature in the step (2) may be 10° C. or lower and 0° C. or higher. This makes it possible to suppress PCR degradation at the time of use after preservation and to suppress degradation due to freezing and thawing of the reaction liquid.

(Aspect 5) In the method according to any one of Aspects 1 to 4, a nucleic acid to be amplified by the PCR reagent may be RNF213 gene p. R4810K polymorphism. This makes it possible to perform a PCR test of RNF213 gene p. R4810K polymorphism and to diagnose atherothrombotic cerebral infarction.

(Aspect 6) In the method according to Aspect 5, the second composition may contain a primer pair for amplifying a base sequence containing a genetic mutation corresponding to the polymorphism, an oligonucleotide fluorescently labeled probe that binds to a mutant-type base sequence containing a genetic mutation corresponding to the polymorphism, and an oligonucleotide fluorescently labeled probe that binds to a wild-type base sequence corresponding to the mutant-type base sequence. As a result, three types (wild-type/wild-type homozygote, mutant-type/mutant-type homozygote, mutant-type/wild-type heterozygote) of positive control results can be obtained, and false negative diagnosis can be reliably suppressed.

(Aspect 7) In the method according to Aspect 2, the PCR reagent may further include a pretreatment liquid, and in the step (2), the pretreatment liquid may be accommodated in a third test container and refrigerated. As a result, the nucleic acid contained in a membrane can be extracted in the specimen, and PCR can be reliably performed. In addition, since a desired amount of the treatment liquid can be added to the third test container in advance, it is not necessary to dispense the pretreatment liquid at the time of specimen collection, and dilution and stirring of the specimen using pipetting can be performed in the third test container. As a result, the PCR test can be started in a shorter preparation time.

(Aspect 8) In the method according to Aspect 7, the pretreatment liquid may be a blood lysate containing an anionic surfactant. With this, the nucleic acid can be extracted from the blood specimen, and PCR can be reliably performed.

(Aspect 9) A PCR test method according to the first embodiment of the present invention includes the steps of performing the preservation method according to any one of Aspects 1 to 6; preparing a liquid from which a nucleic acid is extracted by adding a pretreatment liquid to a specimen; mixing the liquid from which the nucleic acid is extracted with the reaction liquid; and performing PCR on the mixed reaction liquid. As a result, the result of the PCR test can be obtained in a short time.

(Aspect 10) The PCR test method according to Aspect 9, the number of PCR cycles may be 30 or more and 35 or less. With this, the execution time of PCR itself can be further shortened, and for example, the amplification time of PCR can be set to within 45 minutes, and as a result, the time from specimen collection to completion of PCR can be set to within 60 minutes.

### EXAMPLES

Next, the present invention will be described in detail with reference to Examples, but the scope of the present invention is not limited thereto.

### Examples 1 to 4

### (Preservation Step)

A first vial containing BIOTAQ (registered trademark) DNA polymerase (Bioline corporation) and purified water; a second vial containing PCR primer pair represented by SEQ ID NO: 1 (forward) and SEQ ID NO: 2 (reverse), fluorescently labeled probes represented by SEQ ID NO: 3 and SEQ ID NO: 4 (fluorescent dyes may be ROX or FAM, respectively) and purified water; and a third vial containing a dNTP mix, Ampdirect reagent (manufactured by Shimadzu Corporation), KCl, MgCl₂, and a Tris buffer liquid were prepared. In addition, a vial for a blood lysate (pretreatment liquid) containing an anionic surfactant, glycerol, and a Tris buffer liquid was prepared.

Subsequently, the liquid compositions of the first to third vials were mixed to prepare a PCR reaction liquid. The PCR reaction liquid was temporarily stored in a refrigerator at 4° C. for 5 days.

Next, a capped eight consecutive PCR strip tube were prepared, and a part of the reaction liquid was dispensed into the first PCR tube (first test container) together with a wild-type gene of RNF213 gene p. R4810K polymorphism (wild-type/wild-type homozygote: G/G type) as a positive control nucleic acid to prepare a first positive control. A part of the PCR reaction liquid was dispensed into a second PCR tube (first test container) together with a mutant-type gene (mutant-type/mutant-type homozygote: A/A type) of above gene as a positive control nucleic acid to prepare a second positive control. A part of the PCR reaction liquid was dispensed into a third PCR tube (first test container) together with a mutant-type gene of above gene and a wild-type gene (wild-type/mutant-type heterozygote: G/A type) of above gene as a positive control nucleic acid to prepare a third positive control.

Only a part of the PCR reaction liquid was dispensed into a fourth PCR tube to prepare a negative control. Only a part of the PCR reaction liquid was dispensed into fifth to seventh PCR tubes, and these containers were used as specimen test containers (second test containers). That is, three samples to be subjected to the PCR test were prepared. Only the blood lysate (pretreatment liquid) was dispensed into an eighth PCR tube, and this container was used as a pretreatment test container (third test container). At this time, in order to prevent contamination, the strip tube was cut into the first to fourth PCR tubes (A set) and the fifth to eighth PCR tubes (B set) (refer to FIGS. 8 and 9). Thereafter, these eight PCR tubes were capped and further stored in a refrigerator at 4° C. for each desired time (1 hour, 24 hours, 9 days, or 42 days), and then taken out.

### (PCR Step)

Human blood samples (whole blood; National Cerebral and Cardiovascular Center Hospital) was added to the eighth PCR tube, and pipetting was performed to obtain a 10 fold diluted specimen post-treatment liquid. Thereafter, the specimen post-treatment liquid was dispensed into the fifth to seventh PCR tubes to prepare a specimen-containing reaction liquid in which the specimen post-treatment liquid and the PCR reaction liquid were mixed in the fifth to seventh PCR tubes.

Next, the first to seventh PCR tubes were set in a real-time PCR apparatus (LightCycler 96 system, manufactured by NIPPON Genetics Co, Ltd.), and PCR was performed. As setting conditions, after heating at 95° C. for 600 seconds as preincubation, cycles of 10 seconds at 95° C. and 30 seconds at 60° C. were performed 50 times. The total time for PCR was 80 minutes.

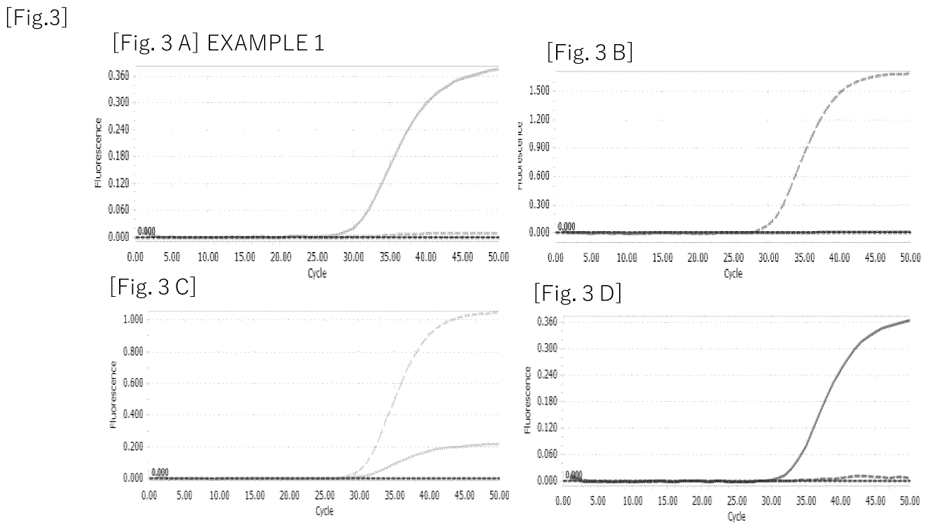

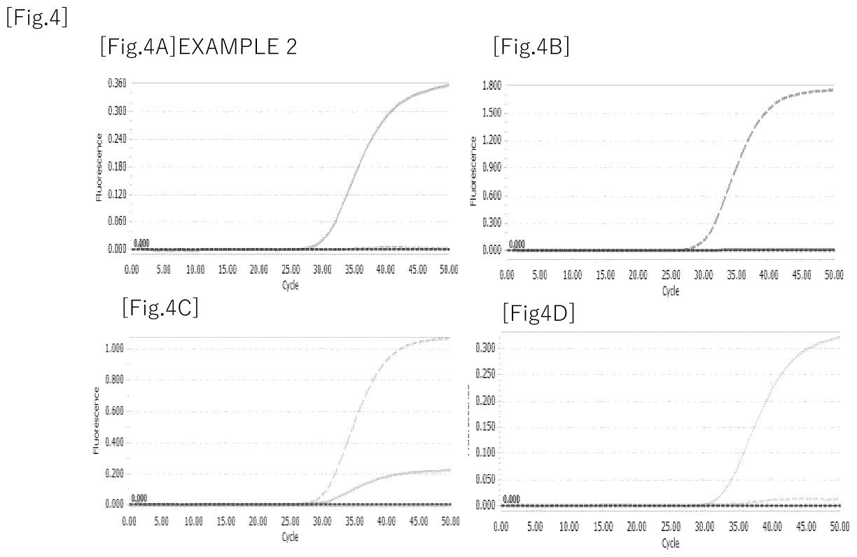

At this time, the PCR results of the first to third and fifth PCR tubes in a case where the storage time after dispensing of the PCR tube is 1 hour are shown in FIG. 3 as Example 1. The PCR results of the first to third and fifth PCR tubes when the storage time is 24 hours are shown in FIG. 4 as Example 2. The PCR results of the first to third and fifth PCR tubes when the storage time was 9 days are shown in FIG. 4 as Example 5. The

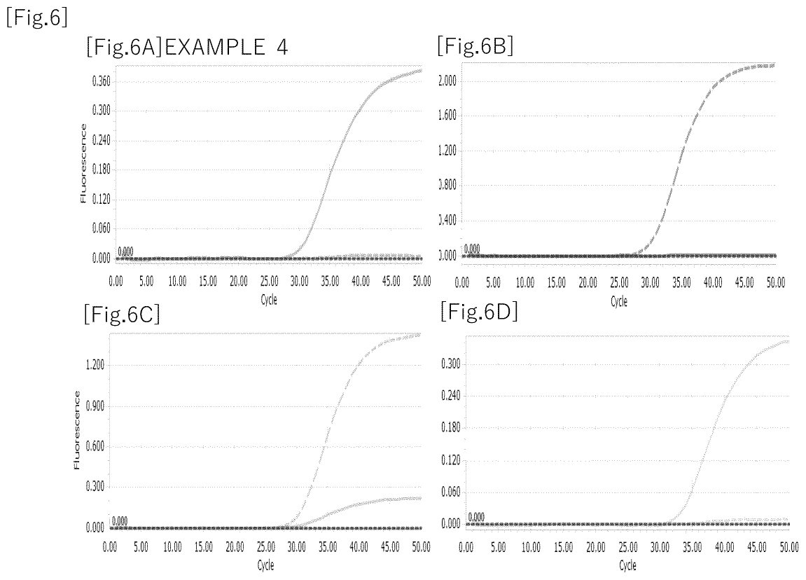

PCR results of the first to third and fifth PCR tubes when the storage time was 42 days are shown in FIG. 6 as Example 4. In each drawings, a solid line represents light emission (FAM) by a fluorescently labeled probe that binds to a wild-type base sequence, and a broken line represents light emission (ROM) by a fluorescently labeled probe that binds to a mutant-type base sequence. In the PCR result of the fourth PCR tube, no rising edge of the amplification curve was found in any of Examples 1 to 4, and the result was negative.

### Reference Example 1

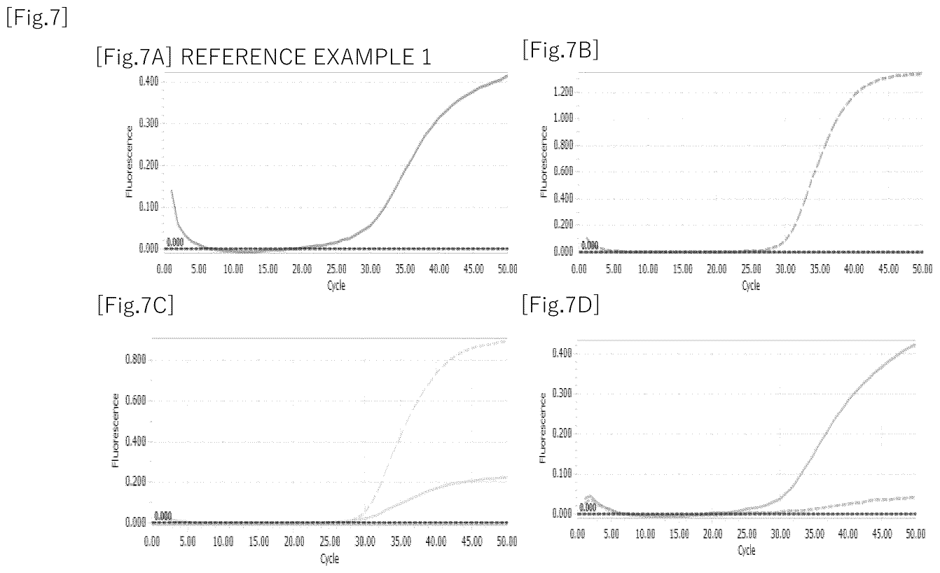

The PCR results in the case of preparing the first to eighth PCR tubes immediately after preparing the PCR reaction liquid and performing PCR are shown in FIG. 7. In the PCR result of the fourth PCR tube, no rising edge of the amplification curve was found, and the result was negative.

From the above results, it was found that since all of Examples 1 to 4 draws an amplification curve almost similarly to Reference Example 1, an appropriate PCR test can be performed even after refrigeration.

### Example 5

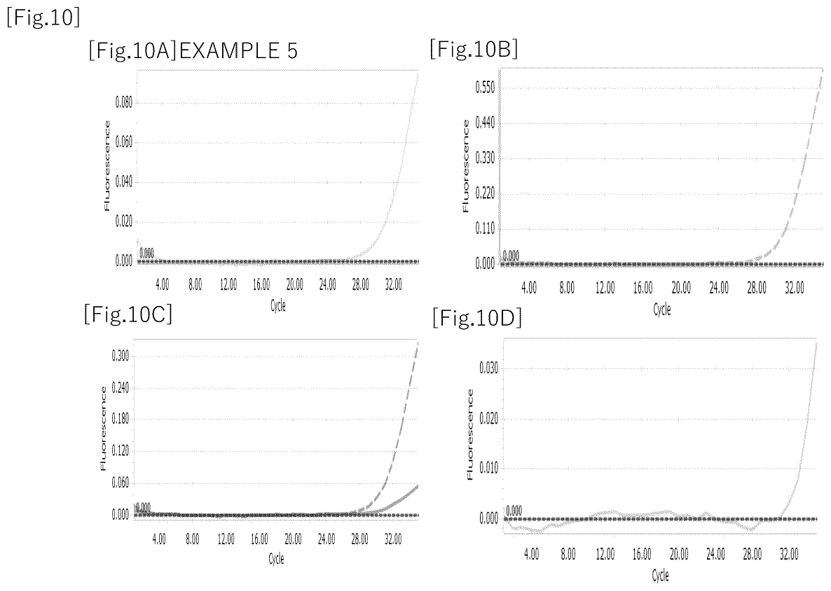

For Example 2, the PCR time was shortened to 45 minutes by changing the pre-incubation to 95° C. for 300 seconds, the PCR cycle to 95° C. for 5 seconds and 60° C. for 15 seconds, and the number of PCR cycles to 35 as PCR conditions. The results are shown in FIG. 10. From this result, it was found that the rising of the amplification curve can be sufficiently confirmed even when the PCR time is 45 minutes, and the amplification curve can be used for determination of the test.

1. A preservation method for a PCR reagent, the method comprising steps of:
(1) mixing all of a first composition containing DNA polymerase, a second composition containing a primer, and a third composition containing a buffer liquid to prepare a reaction liquid; and
(2) refrigerating the reaction liquid,
wherein the first composition, the second composition, and the third composition are accommodated in different containers.

2. The preservation method according to claim 1, wherein
the PCR reagent further includes a positive control, and
in the step (2), a part of the reaction liquid is accommodated in a first test container together with the positive control, and only a part of the reaction liquid is accommodated in a second test container, and then the first and second test containers are refrigerated.

3. The preservation method according to claim 1, wherein the second composition further contains an oligonucleotide probe.

4. The preservation method according to claim 1, wherein a refrigerating temperature in the step (2) is 10° C. or lower, and 0° C. or higher.

5. The preservation method according to claim 1, wherein a nucleic acid to be amplified by the PCR reagent is RNF213 gene p. R4810K polymorphism.

6. The preservation method according to claim 5, wherein the second composition contains a primer pair for amplifying a base sequence containing a genetic mutation corresponding to the polymorphism, an oligonucleotide fluorescently labeled probe that binds to a mutant-type base sequence containing a genetic mutation corresponding to the polymorphism, and an oligonucleotide fluorescently labeled probe that binds to a wild-type base sequence corresponding to the mutant-type base sequence.

7. The preservation method according to claim 2, wherein
the PCR reagent further includes a pretreatment liquid, and
in the step (2), the pretreatment liquid is accommodated in a third test container and refrigerated.

8. The preservation method according to claim 7, wherein the pretreatment liquid is a blood lysate containing an anionic surfactant.

9. A PCR test method comprising the steps of:
performing the preservation method according to claim 1;
preparing a liquid from which a nucleic acid is extracted by adding a pretreatment liquid to a specimen;
mixing the reaction liquid with the liquid from which the nucleic acid is extracted, thereby forming a mixed reaction liquid; and
performing PCR on the mixed reaction liquid.

10. The PCR test method according to claim 9, wherein the a number of PCR cycles is 30 or more and 35 or less.
